(19) United States
(12) Patent Application Publication
(10) Pub. No.: US 2023/0272337 A1
(43) Pub. Date: Aug. 31, 2023

(54) YOLK EXTRACT SUPPLEMENTS FOR CULTURE MEDIA AND RELATED METHODS

(71) Applicant: SUNNYBROOK RESEARCH INSTITUTE, Toronto (CA)

(72) Inventors: Hon S. LEONG, Toronto (CA); Gobi THILLAINADESAN, Ajax (CA)

(21) Appl. No.: 18/012,671

(22) PCT Filed: Jun. 25, 2021
(86) PCT No.: PCT/CA2021/050876

§ 371 Date: Dec. 23, 2022

Related U.S. Application Data
(60) Provisional application No. 63/044,499, filed on Jun. 26, 2020.

(51) Int. Cl. C12N 5/00

(52) U.S. Cl. CPC C12N 5/0037; C12N 2500/80

(57) ABSTRACT

Described herein is an egg yolk extract comprising freed/lysed yolk granules and/or yolk spheres. Also described is an egg yolk extract comprising the liquid contents of a shelled egg, wherein the liquid contents comprise yolk granules. Also described are an egg yolk extract comprising the liquid contents of yolk spheres; an egg yolk extract comprising the liquid contents of yolk granules; an egg yolk extract comprising ovalbumin and other anti-bactericidal proteins in egg white; and an egg yolk extract that supports cell proliferation at least equivalent to FBS when used at the same concentration in a cell culture medium. Related supplements, media, and methods are also described.

### FIELD

The present invention relates to culture media. In particular, the present invention relates to yolk extract supplements for culture media and related methods.

### BACKGROUND

Fetal bovine serum (FBS) is a commonly used growth supplement for cell culture of mammalian cells, as FBS contains various proteins and fats that help the cultured cells remain viable. For many cell lines or cell types, the absence of FBS will lead to cell death and significantly decreased cell viability and lack of cell proliferation. Normally, FBS is used at about 5-10% of the entire volume of the cell culture medium.

FBS is essentially the blood of a bovine fetus that is allowed to clot and then centrifuged to generate serum which is the clear light brown supernatant that is formed after centrifugation. Approximately 3-4 L of whole blood is extracted from each fetus and this is performed while the fetus is present within the mother. The blood is collected via cardiac puncture and is collected into a vessel which is allowed to clot naturally. This process is performed primarily in pregnant milk producing cows. As a result, the cost of FBS is high due to the processes needed and the expenses required for maintenance of these pregnant cows as well as the rising costs of agriculture. In the 2000's a bottle of FBS (500 mL) had a price range of $100-300 (USD). However, in the last ten years, that cost has risen to $300-1,000 USD per bottle. The rising costs of this product mean that eventually the practice of cell culture will be cost prohibitive and could lead to decreased research productivity.

There are alternatives to the use of FBS as a growth supplement but these are predominantly reliant on the purification of proteins that are thought to be required for viable cell cultures. This means that there is no real reduction of the cost for the use of these growth factors and, in some cases, the purified proteins are simply added to diluted FBS. These alternatives as not very popular as they are thought to be synthetic and specific to a cell line and hence not relevant for the culture of a wide variety of cell lines.

Yolk extracts have been used in cell culture as a substitute for FBS. Typically, only the egg yolk is used. The vitelline membrane is pierced and the released egg yolk is collected into tubes for dilution with PBS (saline solution). Once the yolk is diluted (often 5-10× with PBS as the diluent), it is then centrifuged at 200-1,000×g's for 5-20 minutes. After centrifugation, the resulting supernatant is then used for supplementing cell culture studies. These yolk extracts contain only the liquid contents of the yolk and thereby a small fraction of all nutrients present within the entire yolk. When these yolk extracts are used in cell culture studies, they produce a minor growth effect and are inferior to FBS.

For example, U.S. Pat. No. 5,356,798 describes a serum-free medium comprising an egg yolk fraction being free of lipoprotein and lipids. The use of an egg yolk fraction being free of lipoprotein and lipids for increasing the expression of recombinant proteins in a host cell being able to express said protein in a serum-free cell growth medium is disclosed.

Fujii and Gospodarowicz (In Vitro; 19(11):811-817; 1983) describes that supplementation of tissue culture medium with chicken egg yolk can support the proliferation of low density bovine vascular and corneal endothelial cells and vascular smooth muscle cells maintained on basement lamina-coated dishes and that the egg white was devoid of any growth-promoting activity.

Murakami et al. (Cytotechnology; 1:159-169; 1988) describes that egg yolk lipoprotein promoted growth of a wide variety of mammalian cell lines, including plasmacytomas and epithelial cell lines, in serum-free medium. The lipoprotein was characterised as a very low density lipoprotein with a protein content of only 1.3%. This lipoprotein had an optimal concentration of 300 gg/ml (4 gg protein/ml). It was easily separable from proteinous molecules secreted into the serum-free medium by the cells, since it floated on the surface of the medium after addition of ammonium sulfate, to precipitate protein, and centrifugation. An associated structure of lipid and protein seemed to be still necessary for the lipoprotein to exhibit a growth promoting activity.

A need exists for the development of an alternate effective supplement for culture media and related methods.

### SUMMARY OF THE INVENTION

In accordance with an aspect, there is provided an egg yolk extract comprising free yolk granules.

In accordance with an aspect, there is provided an egg yolk extract comprising the liquid contents of an egg yolk and one or more optionally lysed yolk granules.

In accordance with an aspect, there is provided an egg yolk extract comprising the liquid contents of yolk spheres.

In accordance with an aspect, there is provided an egg yolk extract comprising the liquid contents of yolk granules.

In accordance with an aspect, there is provided an egg yolk extract comprising the liquid contents of an egg yolk and one or more egg white proteins.

In an aspect, the one or more egg white proteins comprise ovalbumin, ovotransferrin, lysozyme, ovalbumin-related protein X (OVAX), defensins, ovoinhibitor, AvBD11, or a combination thereof.

In accordance with an aspect, there is provided an egg yolk extract that supports cell proliferation and/or survival at least equivalent to FBS when used at the same concentration in a cell culture medium.

In an aspect, the extract comprises free yolk granules In an aspect, the extract comprises lysed yolk spheres.

In an aspect, the extract comprises one or more egg white proteins such as ovalbumin, ovotransferrin, lysozyme, ovalbumin-related protein X (OVAX), defensins, ovoinhibitor, AvBD11, or a combination thereof.

In an aspect, the extract comprises the homogenized yolk and white of an egg.

In an aspect, the egg yolk extract consists essentially of egg yolk and egg white, wherein the egg yolk comprises lysed yolk spheres.

In an aspect, the egg yolk extract is liquid.

In an aspect, the egg yolk extract is extracted from an egg of any oviparous species, for example, birds, reptiles, amphibians, fish, insects, molluscs, arachnids, or combinations thereof.

In an aspect, the egg yolk extract is avian.

In an aspect, the egg yolk extract is extracted from an egg of a chicken, turkey, duck, goose, quail, pheasant, ostrich, emu, or combinations thereof.

In an aspect, the egg yolk extract is chicken, duck, or quail.

In accordance with an aspect, there is provided a supplement for cell culture medium, the supplement comprising the egg yolk extract described herein.

In an aspect, the extract consists essentially of the egg yolk extract described herein. In an aspect, the extract consists of the egg yolk extract described herein.

In an aspect, the yolk extract is not diluted.

In an aspect, the yolk extract is not diluted with saline, such as PBS.

In accordance with an aspect, there is provided a cell culture medium comprising the egg yolk extract or the supplement described herein.

In an aspect, the medium is Dulbecco's Modified Eagle Medium (DMEM), RPMI media, CMRL1066, Hanks' Balanced Salt Solution (HBSS) phosphate buffered saline (PBS), L-15 medium, DMEM-F12, EpiLife® medium, and Medium 171, or a combination thereof.

In an aspect, the medium is a buffered saline solution, such as HBSS.

In an aspect, the medium comprises from about 1% to about 10% v/v of the extract or supplement, such as about 1%, about 2%, about 3%, about 4%, about 5%, about 6%, about 7%, about 8%, about 9%, or about 10%.

In an aspect, the cell culture medium does not contain FBS and/or NCS.

In an aspect, the cell culture medium is a growth medium or a freezing medium, for example a freezing medium without DMSO.

In an aspect, the cell culture medium is for culturing primary cell lines, for example, primary cell lines for food consumption, for example, primary bovine skeletal muscle cells.

In an aspect, the cell culture medium is for use in culturing cells for antibody production.

In an aspect, the cell culture medium is for use in biologics production, for example, biologics such as insulin, erythropoietin (EPO), or Granulocyte Colony Stimulating Factor (C-GSF).

In accordance with an aspect, there is provided a method for making an egg yolk extract, the method comprising lysing yolk spheres.

In an aspect, lysing yolk spheres comprises sonicating or homogenizing the yolk spheres, for example by high pressure homogenization.

In an aspect, the method is sufficient to lyse at least about 50%, about 60%, about 70%, about 80%, about 90%, about 95%, or about 99% of the yolk spheres.

In an aspect, the sonicating is at about 75 W or more for from about 25 to about 30 cycles at about 15 to about 20 seconds per cycle.

In an aspect, the yolk spheres are in a composition comprising egg yolk and egg white.

In an aspect, the method further comprises mixing the egg yolk and egg white before lysing the yolk spheres, optionally wherein the mixing is for from about 30 minutes to about 24 hours, at from about 4° C. to about 25° C., such as for about 30 minutes at room temperature or for about 12 to 18 hours at 4° C., and optionally wherein the mixing is by inversion.

In an aspect, the method further comprises removing the vitelline membrane from the egg yolk before or after mixing the egg yolk and egg white.

In an aspect, the method further comprises dissolving unlysed yolk spheres and/or yolk granules for example by alkalinizing and/or salinating the lysed yolk spheres.

In an aspect, alkalinizing and/or salinating the lysed yolk spheres comprises adding a base and/or salt to the lysed yolk spheres, such as a strong base, such as NaOH (optionally at from about 100 mM to about 1 M), KOH (optionally at from about 100 mM to about 1 M), Na₂SO₄ (optionally at from about 0.05 mM to about 0.1 M), (NH₄)₂SO₄ (optionally at from about 0.05 mM to about 0.5 M), or a combination thereof or such as NaCl, optionally at from about 0.1 M to about 0.5M.

In an aspect, the method further comprises removing solids.

In an aspect, removing solids comprises centrifuging the lysed yolk spheres, for example at from about 15,000 g to about 30,000 g, and retaining the supernatant.

In an aspect, the method further comprises sterilizing the egg yolk extract, for example, using a filter with a pore size of 0.4 μm or smaller, such as about a 0.22 μm or 0.11 μm pore size.

In an aspect, the method further comprises clarifying the egg yolk extract, for example by adding glycerol at from about 50 to about 200 μL per 1 mL of egg yolk extract.

In accordance with an aspect, there is provided an egg yolk extract made by the method described herein.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIG. 1. Sonication Results in Yolk Sphere Pellet Homogenization and Superior Growth Effect When Used in Cell Culture of MDA-MB-231 cells. A) Visual inspection of a non-sonicated yolk+egg white (left tube) and sonicated yolk+egg white (right tube) after centrifugation at 14K RPM. The pellet is composed of Yolk Spheres, which are very large cells (30-70 μm in diameter) that are the main cell type within the Yolk itself. Yolk Spheres contain an abundance of lipid granules and yolk granules (both are smaller than 2 μm in diameter) that are rich in nutrients such as fatty acids, proteins, amino acids, vitamins, various metabolites, and glucose. Sonication breaks open the majority of these yolk spheres, as evidenced by the smaller pellet in A) and B). The bar graph represents the amount of yolk sphere pellet generated after sonication and subsequent centrifugation (14K RPM) of each yolk as treated by sonication or lack of (N=3, error bars are SEM). C) Yolk+egg white were submitted to no sonication, moderate sonication (3×, 1 minute each cycle), and long sonication (25×, 1 minute each cycle). After processing to generate an extract described herein as “Complete Yolk Extract” (CYE, which comprises taking yolk+egg white and submitting it to sonication+chemical treatment, centrifugation, filtration), each preparation was used to supplement DMEM for MDA-MB-231 cell culture. Cells were cultured for 3 days and then cells were counted at the end of that time-course experiment. The highest number of cells were observed in the “long sonication” yolk+egg white preparation. This homogenization process results in the highest amount of nutrients present in the CYE and produced the largest cell growth effect in C).

FIG. 2. Comparison of Complete Yolk Extract (CYE) and Fetal Bovine Serum (FBS) at Normal Culturing Conditions. MDA-MB-231 cells were cultured in vitro with DMEM and CYE (labeled as Yolk Extract) or FBS at either 3% or 5%. Cells were seeded and then cultured for 3 days. At the end of 3 days, representative images were acquired (4× microscope objective) and then cells were harvested and counted. The images reveal cells that are larger and healthier when treated with CYE compared to FBS. Healthy dense monolayers were also observed with CYE treatment compared to FBS treatment. The bar graph reveals the growth effect of each type of growth supplementation. Notably, the 3% CYE condition exhibited a superior growth effect compared to the 3% FBS condition and an equivalent growth effect compared to the 5% FBS condition.

FIG. 3. MDA-MB-231 Cells Cultured with Various CYE/FBS Concentrations. Images of cultured cells (4× microscope objective) were obtained when cells were cultured with DMEM and the specified concentration of Chicken Complete Yolk Extract (labeled as Chicken Yolk), Duck Complete Yolk Extract (labeled as Duck Yolk), and FBS. Control represents DMEM only. Three different Duck Complete Yolk Extracts were prepared and compared between each other at the given concentrations. The majority of all CYE or FBS supplemented cell culturing conditions yielded near confluent cell monolayers at the end of the 3 day experiment.

FIG. 4. Consistency of CYE Batches on Cell Growth. MDA-MB-231 cells were cultured in DMEM for 3 days with the specified concentration of growth factor (CYE/FBS). Two different batches of CYE were used for this experiment. The number of cells were higher in both CYE supplemented media compared to FBS in the 3% condition. The number of cells present between the two CYE batches were similar, revealing consistency between CYE batches. The images (4× microscope objective) reveal healthy dense monolayers of CYE (10%) treated cells that reach full confluence within 3 days.

FIG. 5. DU145 Cells Cultured with DMEM Supplemented with Various Concentrations of Complete Yolk Extract (CYE). Images of DU145 cells grown under the specified condition (0-10% CYE) taken after 3 days of culture. The cells in all growth conditions are healthy, but the highest confluency is observed in any treatment with CYE (1% to 10% CYE).

FIG. 6. Complete Yolk Extract (CYE) is Superior to Fetal Bovine Serum (FBS) in PC3 Cultured Cells. A) PC3 cells were cultured for 3 days with various concentrations of CYE or B) FBS. C) Number of cells grown under each culturing condition (0, 1, 2, 5, 7.5, 10% of CYE or FBS) reveals a growth advantage with CYE. The largest difference was observed with 1-2% CYE growth supplementation.

FIG. 7. Comparison of Complete Yolk Extract (CYE) and Fetal Bovine Serum (FBS) and their Effect at Low Concentrations on Cell Growth. MDA-MB-231 cells were cultured in DMEM with either 1% FBS or 0.6% CYE for 3 days. At the end of 3 days under these low growth factor conditions, the 0.6% CYE condition yielded more cells than the 1% FBS condition as evidenced by more adherent cells in this representative field of view (4× microscope objective).

FIG. 8. Complete Yolk Extract (CYE) Is Able to Maintain Cell Viability Under Low Concentrations. To reveal the effectiveness of CYE, HepG2 cells (top panels) and MDA-MB-231 cells (bottom panels) were cultured in DMEM without any CYE (Control 0%; left side of panels) or cultured in DMEM with a low amount of CYE (0.4%). After three days of growth, representative images were acquired (4× microscope objective) of adherent cells. The HepG2 cells received some growth benefit when cultured with 0.4% CYE. However, MDA-MB-231 cells were not viable in control conditions (0% CYE) but were viable and adherent in 0.4% CYE supplemented culturing conditions.

FIG. 9. Long Term Culture with CYE Preserves MDA-MB-231 Cell Viability. Different CYE preparations were generated by varying the centrifugation speed spin (post-sonication) and storage of the preparation prior to use in cell culture. Cells were cultured with 5 different 3% CYE “preps” (14/7K RPM of centrifugation for 15 minutes; +4° C./−20° C. storage of CYE prep overnight prior to use) or no supplementation (0%). Cells were left in culture for 10 days with no media changes. Images were obtained (4× microscope objective) of each culturing condition. Bar graph represents the number of cells present after the 10 day culture. The 3% CYE group represents all of the 5 culturing conditions with 3% CYE (100%+4° C.; 14K Spin −20° C.; 14K Spin +4° C.; 7K Spin −20° C.; 7K Spin +4° C.). Error bars represent SEM.

FIG. 10. Complete Yolk Extract (CYE) Does not Require DMEM Media and Can Be Replaced with Hanks Buffered Saline Solution (HBSS). Different combinations of media (PBS, DMEM, HBSS) were used with CYE (3% Yolk Extract) to culture MDA-MB-231 cells for 2 days. Images were acquired (4× microscope objective) of cells under each growth condition. Cells grown with HBSS and CYE appeared to be as viable as cells grown with DMEM and CYE.

FIG. 11. HBSS+5% CYE Outperforms DMEM+5% FBS In MDA-MB-231 Cell Culture Growth Rates. A) Representative image (4× microscope objective) taken of MDA-MB-231 cells grown with HBSS+5% CYE. B) Representative image (4× microscope objective) taken of MDA-MB-231 cells grown with DMEM+5% FBS. C) Cell number after 3 days of culture revealing equal or greater than growth rates with the HBSS+CYE combination compared to DMEM+FBS.

FIG. 12. Complete Yolk Extract (CYE) Decreases Bacterial Contamination. CYE was tested for its ability to inhibit bacterial contamination in cell culture. Cell cultures were exposed to ambient air overnight to induce bacterial contamination (open). The OD of media cultured for 48 hours in 37° C. was determined and the ratio of ODopen/ODclosed was determined for various groups. Supplementation was with 5% FBS or 5% CYE. A decrease in ratio ODopen/ODclosed was observed in the CYE+Pen/Strep group and the CYE alone group (n=3/group) when compared to their FBS counterparts.

FIG. 13. Complete Yolk Extract (CYE) is as Effective as DMSO as a Cryo-Preserving Solution. Various freezing media/cyro-preservant solutions were tested: A) DMEM only; B) 10% DMSO, 90% DMEM; C) 10% DMSO, 90% FBS, D) CYE only; E) FBS only. After cryo-preservation with A-E) for 48 hours, MDA-MB-231 cells were thawed and cultured in DMEM+10% FBS for 24 hours. Representative images are shown of the number of viable and adherent cells after cryo-preservation with each type of freezing media. The CYE only freezing media resulted in a high number of adherent and viable cells after thawing and plating into well filled with cell culture media.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

Complete Yolk Extract (CYE) Described herein is a complete yolk extract (CYE) that can be used in cell culture media, for example as a substitute for FBS. The CYE described herein comprises a homogenized mix of egg white (albumin) and egg yolk, typically from shelled eggs. It will be understood that the term “complete” does not necessarily imply that the extract contains all the components of an egg or egg yolk but, rather, that the extract comprises nutrients generally considered sufficient for cell survival and/or proliferation. The nutrients present within the egg yolk and egg white, including ovalbumin, are excellent for the sustenance of cultured cells. The yolk is a rich source of proteins, amino acids, glucose, fatty acids, complex lipids, vitamins, and various metabolites consumed by the embryo as it grows in isolation within its egg shell. The ovalbumin in egg white is functionally similar to albumin (in humans or cows) which is a key component of blood and is abundant in FBS; both are a protein source for cell culture. When the egg yolk and egg white are processed as described herein, the resulting liquid can be used to supplement cell culture media for growing and/or expanding cells needed for research and/or the manufacturing of biotechnology products (vaccines, antibodies, biologics/drugs). The data shown below demonstrate that the CYE described herein consistently delivers adequate cell growth and proliferation results that are, in aspects, equal or superior to FBS.

The production of CYE involves processing of the natural components of eggs, such as avian eggs (egg white and egg yolk) to arrive at an aqueous and easy-to-use liquid product that can support cell growth without additional supplementation by other growth factors. This processing removes the membranous barriers that separate key components of an egg, such as the albumin (egg white) and the egg yolk. As described herein, these barriers are physically and/or chemically removed in order to release their liquid contents and nutrients contained therein. For example, the vitelline membrane separates the egg yolk from the egg white. Within the egg yolk, there exists millions of large cells called yolk spheres (30-70 μm in diameter) that contain a highly concentrated amount of fat, vitamins, and protein. In contrast, the liquid contents of the yolk excluding the yolk spheres contains a much smaller concentration of fat, vitamins, and protein. In addition, there are yolk granules (vesicles that are 1-5 μm in diameter) that also contain various nutrients not found in the other components such as specific proteins, and are also useful for cell viability and cell proliferation. Yolk granules can be found within the egg yolk and are also found within Yolk Spheres which are 30-70 μm in diameter. The processes described herein release the contents of yolk spheres and yolk granules, and homogenize all of these previously-compartmentalized nutrients into a single liquid extract. This single liquid extract is then used in the same manner as FBS but typically at a lower concentration due to the much higher density/concentration of nutrients present in CYE.

Thus, in aspects, described herein is an egg yolk extract comprising free yolk granules. In other words, the egg yolk extract comprises yolk granules that have been released from or are no longer contained in yolk spheres. In aspects, the egg yolk extract (CYE) describe herein comprises the liquid contents of yolk granules and/or comprises lysed yolk granules. In other aspects, the egg yolk extract described herein comprises the liquid contents of yolk spheres. In some aspects, the egg yolk extract comprises the liquid contents of both yolk spheres and yolk granules and further optionally comprises free yolk granules and/or lysed yolk granules.

In aspects, described herein is an egg yolk extract comprising the liquid contents of an egg, such as a shelled egg, wherein the liquid contents comprise yolk granules. The yolk granules may be whole or they may be lysed, or there may be a combination of whole and lysed yolk granules in the egg yolk extract.

In additional or alternative aspects, described herein is an egg yolk extract comprising ovalbumin and other protein components of the egg white, such as ovotransferrin, lysozyme, ovalbumin-related protein X (OVAX), defensins, ovoinhibitor, AvBD11, or combinations thereof. Further described herein is an egg yolk extract that supports cell proliferation at least equivalent to FBS when used at the same concentration in a cell culture medium.

In aspects the egg yolk extracts described herein comprise the homogenized yolk and white of an egg. Typically, the egg and yolk are mixed together, as described below, and subsequently homogenized in order to release the nutrients from the yolk spheres and/or yolk granules and will be mixed with other nutrients in the egg white. These nutrients are released and combined into the liquid mixture, making these nutrients more accessible to cells growing in a culture medium containing the egg yolk extract.

It will be understood that while the egg yolk extract may comprise the homogenized yolk and white of an egg it may instead consist essentially of the homogenized yolk and white of an egg or consist solely of the homogenized yolk and white of an egg. The egg white and yolk may be derived from the same egg or from different eggs of the same species or from different eggs of different species. Moreover, the egg white and egg yolk may be pooled from a plurality of eggs at different ratios. For example, there may be included one egg white for every 1-10 egg yolks or one egg yolk for every 1-10 egg whites.

The egg yolk extract described herein may be from any one or a combination of oviparous species, for example, birds, reptiles, amphibians, fish, insects, molluscs, arachnids, or any combination thereof. Typically, the egg yolk extract is avian and is, for example, from a chicken, turkey, duck, goose, quail, pheasant, ostrich, emu, or any combination thereof. Typically, the egg yolk extract described herein is from a chicken, duck, or quail.

Typically, the egg yolk extract described herein is liquid, however, it may be provided in other forms such as a dried (lyophilized or spray-dried, for example) form.

Also described herein is a supplement for cell culture medium that comprises the egg yolk extract. The supplement may consist essentially of the egg yolk extract or it may consist only of the egg yolk extract. It will be understood that the egg yolk extract described herein is considered “complete” in that it provides sufficient nutrients to support the growth and/or survival of cells, such as mammalian cells, avian cells, yeast cells, bacterial cells, etc.

In typical aspects, the yolk extract supplement is not diluted prior to being added to a culture medium. For example, the egg yolk extract described herein is not mixed with any substantial amounts of saline, such as PBS. Such non-diluted egg yolk extract may be described as being full strength for example.

Also described herein are media for cell culture comprising the egg yolk extract or the supplement described above. Any medium is contemplated herein. Examples include Dulbecco's Modified Eagle Medium (DMEM), RPMI media, CMRL1066, Hanks' Balanced Salt Solution (HBSS) phosphate buffered saline (PBS), L-15 medium, DMEM-F12, EpiLife® medium, and Medium 171. Typically, the medium is DMEM or a buffered saline solution, such as HBSS.

The cell culture medium may contain the supplement or extract in any suitable amount. For example, the medium may comprise from about 1% to about 10% v/v of the extract or supplement, such as about 1%, about 2%, about 3%, about 4%, about 5%, about 6%, about 7%, about 8%, about 9%, or about 10% of the extract or supplement.

The extract and supplement described herein is a suitable replacement for commonly used supplements such as FBS or NCS. Thus, in aspects, the cell culture medium described herein does not contain FBS and/or NCS. In aspects, the cell culture medium described herein is serum-free. The cell culture medium in aspects may contain FBS and/or NCS and it will be understood that other components normally present in cell culture medium may be included in the media described herein. For example, antibiotics may be included.

The cell culture medium described herein may be used for any purpose. For example, the medium may be a growth medium or a freezing medium, for example a freezing medium without DMSO. In conventional freezing media comprising DMSO, there is often cell loss during the thawing process as the DMSO is cytotoxic. The cells need to be thawed quickly and carefully to ensure viability. Freezing media comprising the extract or supplement described herein, where DMSO is absent or at a reduced concentration, typically results in an easier thaw process with less cell loss than when conventional freezing media such as DMSO is used.

In specific aspects, the cell culture medium is for culturing primary cell lines, for example, primary cell lines for food consumption, for example, primary bovine skeletal muscle cells. It will be understood that the cell culture media described herein may find particular use in the culture of lab grown meat, for example. Typically, in order to grow lab grown beef, the culture medium used needs to be supplemented with FBS, which of course is sourced from fetal cows. This means that lab grown beef still relies heavily on the bovine livestock industry. Using a cell culture medium that can support the growth of lab grown meat without relying on living cows is a clear advantage of the cell culture media, supplements, and extracts described herein.

In other aspects, the cell culture medium described herein finds use in culturing cells, such as yeast cells, bacterial cells, or mammalian cells, such as hybridoma cells, in biotechnology applications. For example, for antibody production on a mass commercial scale could benefit from the use of the cell culture media, supplements, and extracts described herein. Culturing hybridoma cell lines with the extract, supplement, or medium described herein would enable the mass production of IgG/IgM antibodies without risk of isolating any contaminant bovine IgG/IgM proteins. Such contaminant proteins (e.g., bovine IgG) are abundant in FBS but not present in egg yolk extract. The IgY protein found in egg yolk extract is typically not isolated during drug/antibody purification, thus accelerating and/or simplifying drug/antibody production.

### Methods

Also described herein are methods of making an egg yolk extract. Typically, the method comprises lysing yolk spheres. The yolk spheres may be lysed or broken open by any known method, for example by sonicating and/or homogenizing the yolk spheres, for example by high pressure homogenization. When sonication is used, it is typically at about 75 W or more for from about 25 to about 30 cycles at about 15 to about 20 seconds per cycle, for example, when about 25 mL is being sonicated. The skilled person will appreciate that these sonication conditions can be modified for different volumes.

While the yolk spheres may be processed without other egg components, typically the yolk spheres are in a composition comprising other egg components, such as the liquid contents of the egg yolk and/or egg white. Typically, the method comprises mixing the egg yolk and egg white and then lysing the yolk spheres, for example by sonicating the mixture. The egg white and egg yolk may be mixed by any method but, typically an inverter is used. The mixing is typically for a period of from about 30 minutes to about 24 hours, at from about 4° C. to about 25° C. It will be understood that typically longer time periods of mixing are done at about 4° C. For example, the mixing may be for about 30 minutes at room temperature or for about 12 to 18 hours at 4° C.

In aspects, the method comprises removing the vitelline membrane from the egg yolk before or after mixing the egg yolk and egg white.

The lysing of the yolk spheres may be improved by alkalinizing and/or salinating the lysed yolk spheres before, after, and/or during the lysing step, such as sonication, described above. Typically, alkalinizing and/or salinating the lysed yolk spheres comprises adding a base and/or salt to the lysed yolk spheres, such as a strong base, such as NaOH (optionally at from about 100 mM to about 1 M), KOH (optionally at from about 100 mM to about 1 M), Na2 SO4 (optionally at from about 0.05 mM to about 0.1 M), (NH4)2SO4 (optionally at from about 0.05 mM to about 0.5 M), or a combination thereof or such as NaCl, optionally at from about 0.1 M to about 0.5M.

Once the lysing is complete to the desired level, solids may be removed from the composition, leaving the liquid contents behind. Typically, solids are removed by centrifuging the lysed yolk spheres, for example at from about 15,000 g to about 30,000 g, and retaining the supernatant. The supernatant can be used as the egg yolk extract cell culture supplement at this stage, or it may be sterilized first, for example, filter-sterilized, typically using a filter with a 0.22 μm pore size.

If desired, the egg yolk extract may be clarified or made more clear by adding glycerol, which is optionally sterile, at from about 50 to about 200 μL per 1 mL of egg yolk extract.

Once prepared, the egg yolk extract may be used as desired, typically as a cell culture supplement as described herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way in the remainder of the disclosure.

### EXAMPLES

### Example 1: Preparation of Complete Yolk Extract (CYE)

The CYE described herein is typically comprised of the entire yolk and egg white processed generally as outlined below. The resulting liquid product when used as a cell culture supplement in lieu of FBS produces cell growth and proliferation rates that are in aspects superior to FBS when compared at the same concentrations or lower. The CYE product also does not require dilution with PBS, which is important since it will reduce dilution effects when adding growth supplements. The general processing steps to prepare CYE are as follows:
1. Obtain both the egg white (albumin) and egg yolk from a cracked egg.
2. Remove the vitelline membrane from the egg yolk and then combine the egg white and the yolk.
3. Mix the white+yolk mixture for 30 minutes at room temperature or alternatively overnight at 4° C. for better results.
4. Sonicate of the white+yolk mixture at >75 Watts for 15-20 seconds (1 cycle) for 25-30 cycles. Cooling the mixture during sonication helps reduce overheating the mixture.
5. Addition of 10 μL of 1M NaOH to further dissolve any un-lysed Yolk Spheres and Yolk Granules (1-5 μm in diameter) present within Yolk Spheres (30-70 μm in diameter).
6. Centrifugation of this mixture at 30,000 g's 14K rpm at room temperature for 15 minutes. The resulting pellet will be significantly smaller than the un-sonicated pellet.
7. Reserve the supernatant and filter sterilize with a 0.22 μm pore filter.
8. Add Sterile Glycerol (100% stock, 10-200 μL per every 1 mL of filtered supernatant)
9. This filtered supernatant is “Complete Yolk Extract” or CYE and can be used as a cell culture supplement in the same use or manner that FBS is used.

### Example 2: Testing of Complete Yolk Extract (CYE)

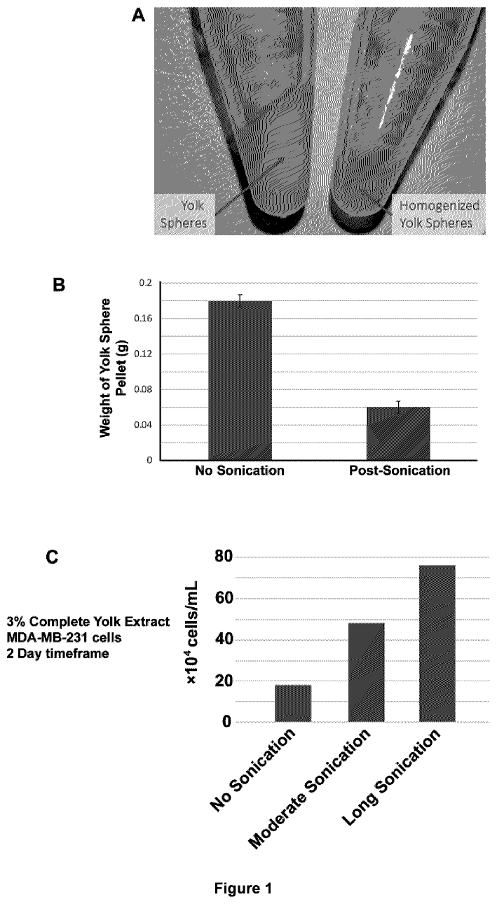

In the first experiment, we demonstrated the importance of sonicating the egg mixture (egg white+yolk) in order to liberate the nutrients housed within the Yolk Spheres and their granules. In FIG. 1A, sonication of the egg mixture resulted in lysis of Yolk Spheres and yielded a smaller pellet after centrifugation (Step #6 above) compared to egg mixture that was not sonicated. These pellets were weighed and are shown in FIG. 1B. When the supernatant was filtered and used for cell culture of MDA-MB-231 cells, the results of that cell growth experiment are presented in FIG. 1C. We observed a significant increase in cell numbers after incubation of complete yolk extract/CYE (long sonication, right bar), and with yolk extract that had mild sonication (middle bar). In contrast, yolk extract that received no sonication (left bar) yielded the lowest numbers of cell proliferation over the same time frame. Hence, we determined that liberation of the nutrients present in the Yolk Spheres leads to greater cell proliferation upon treatment with this Complete Yolk Extract (CYE). Lack of sonication yielded a yolk extract that was not beneficial for cell growth and proliferation.

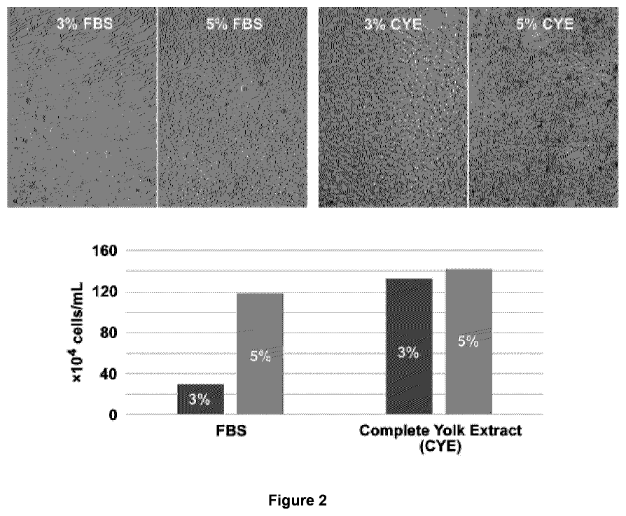

In the second experiment, we compared the growth effect of CYE versus FBS when used to culture MDA-MB-231 cells over a 3 day time frame. The use of 3% CYE resulted in significantly higher cell proliferation rates compared to 3% FBS (FIG. 2 bar graph). Cells fed with CYE also appeared to be healthier in terms of morphology and healthier when they formed cell monolayers when the plates reached full confluency (FIG. 2 upper panels of images). This head to head experiment demonstrated that CYE has a superior growth effect at lower concentrations when compared to FBS. To our knowledge, this has not been demonstrated previously by another type of “yolk extract”.

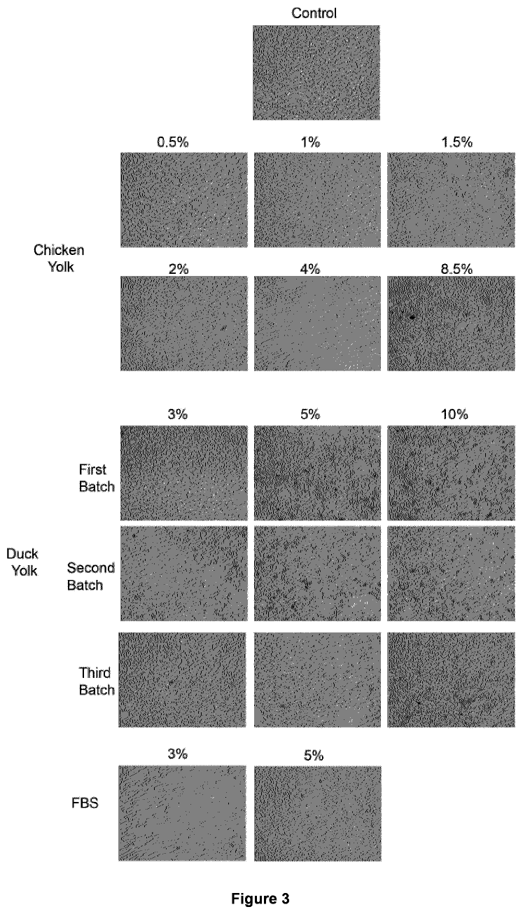

In the third experiment, we looked at two different types of eggs; those from chickens and those from ducks. From these experiments (FIG. 3), we showed that both types of eggs led to CYE that yielded excellent growth effects on MDA-MB-231 cell cultures. We also show that these cells appear to be healthier either at lower confluency or at high confluency when they form cell monolayers. We also found minimal/decreased cell proliferation when cells were cultured with no growth factor supplementation (Control image in FIG. 3). This is a very important negative control since it emphasizes the need for growth factor supplementation for this cell line.

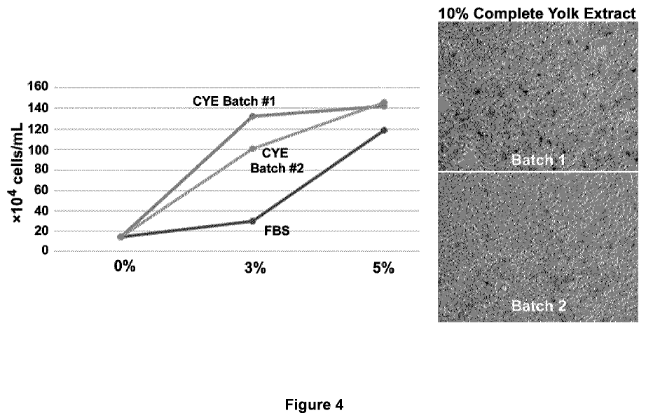

To demonstrate the consistency between batches of CYE (from duck eggs), we counted the number of cells that grew/proliferated over the same time frame (same data as FIG. 3) when treated with Batch #1, Batch #2 (both CYE) and FBS. The same 3% and 5% concentrations were used for the CYE batches and FBS. As shown in FIG. 4, the growth/proliferation rates are significantly higher than FBS at 3% and 5% and no major difference between CYE Batch #1 and #2 is observed. The images on the left for 10% CYE are the same representative images used in FIG. 3.

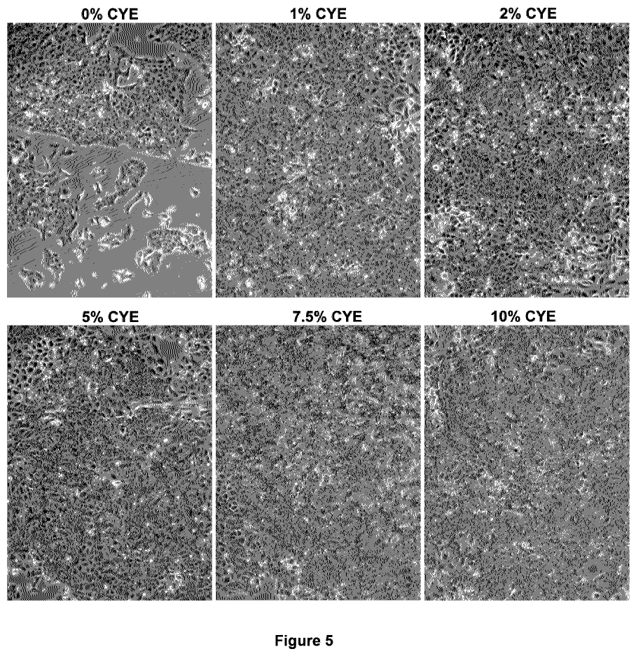

DU145 cells are a cell line representing invasive prostate adenocarcinoma and are androgen-insensitive (do not require testosterone for growth). This cell line was cultured with various concentrations of CYE (0, 1, 2, 5, 7.5, 10% CYE) revealing excellent and rapid growth over 3 days (FIG. 5). This cell line was used because it does remain viable without the need for growth factors when cultured with no growth factor supplementation (0%).

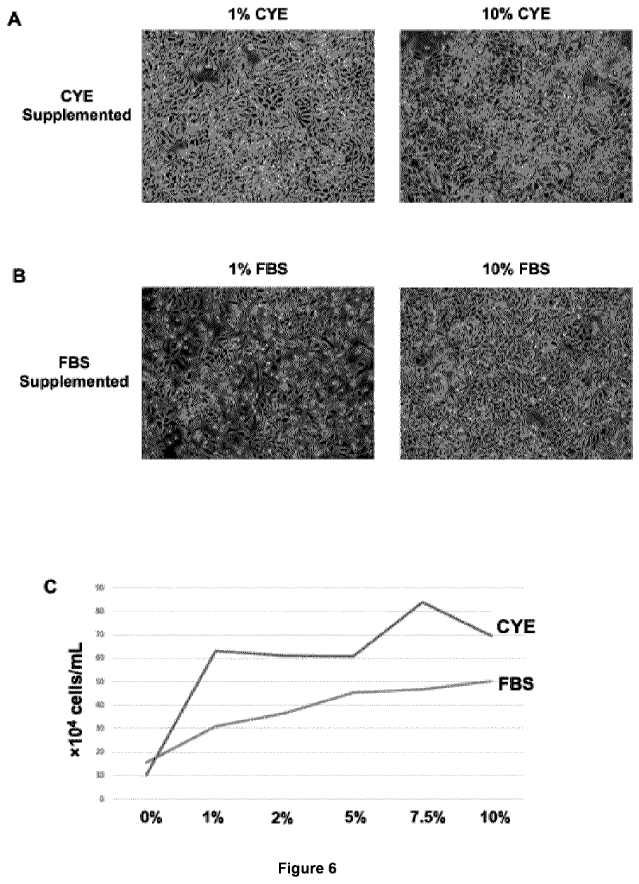

Another prostate cancer cell line, PC-3, was also used and CYE was compared to FBS at various concentrations. In FIG. 6A, a higher number of PC-3 cells were present at the 1% CYE concentration relative to its 1% FBS equivalent (FIG. 6B). After three days of growth, treatments with >3% CYE/FBS lead to near full confluency as shown in the right panel of images in FIG. 6A-B. In FIG. 6C, CYE demonstrated higher numbers of PC-3 cells at all concentrations used compared to FBS except for Control (0%). Hence, CYE is superior to FBS in terms of cell proliferation at various concentrations of growth factor supplementation used for PC-3 cells.

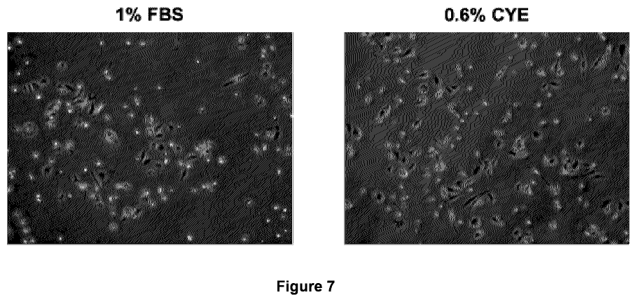

Many laboratories attempt to culture cells at lower concentrations of growth factor supplements, with increasingly more experiments using 5% FBS instead of 10% FBS. To demonstrate the effectiveness of CYE at low concentrations, MDA-MB-231 cells were cultured in 0.4% CYE or 1% FBS. These images reveal that a lower concentration of CYE yielded more cell proliferation than 1% FBS (FIG. 7). These results suggest that CYE can be used to culture cells at low growth factor conditions and is superior to FBS.

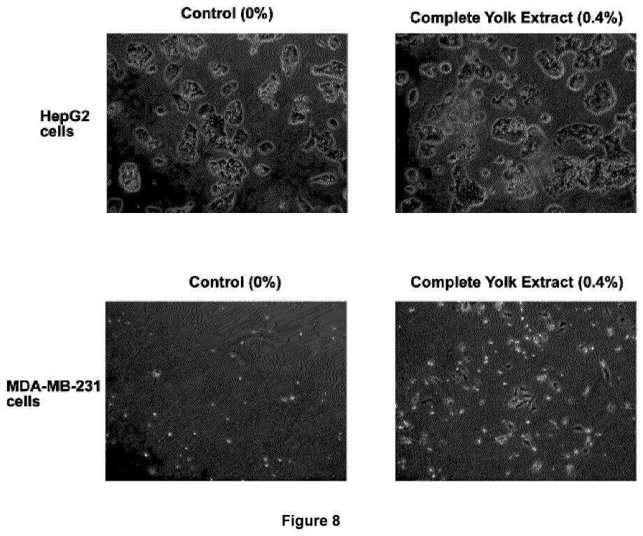

To demonstrate that this low growth factor condition applies to other cell lines, we performed the same experiment in HepG2 cells with MDA-MB-231 cells as a control (FIG. 8). A positive cell growth effect was also observed at low concentrations of CYE (0.4%) compared to control (0%). MDA-MB-231 cells cultured in the absence of CYE (0%) had negligible cell viability after 3 days of culture and presence of viable cells at 0.4% CYE.

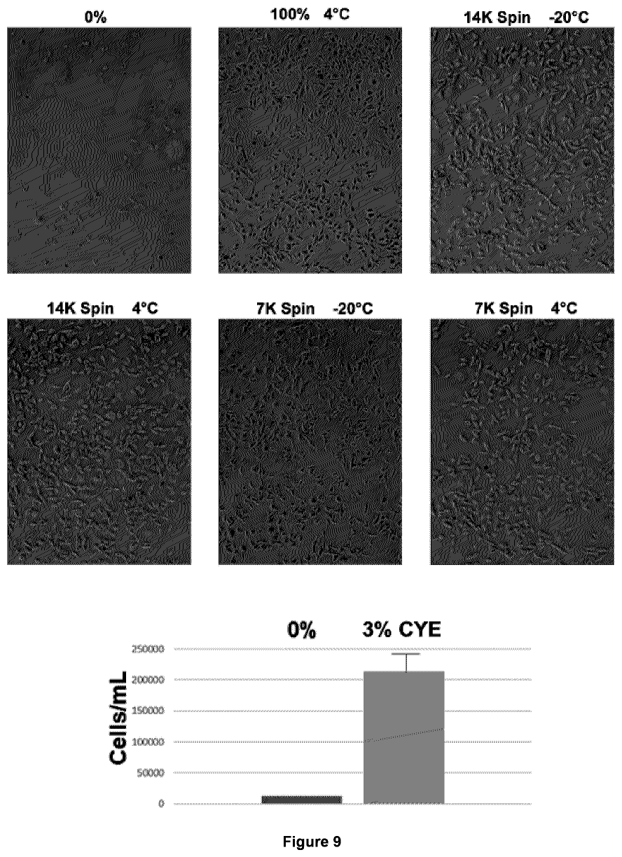

A long term culture of MDA-MB-231 cells was performed in which cell culture media was not changed for 10-11 days. In this experiment, control treated cells (0% CYE) led to negligible viable cells at endpoint. However, various different CYE preparations (centrifuge speed between 14K/7K rpm, overnight storage at 4/−20° C.) that were used at 3% all resulted in significantly higher viable cells present at endpoint (FIG. 9). These results suggest that the CYE is able to provide long term culturing of cells.

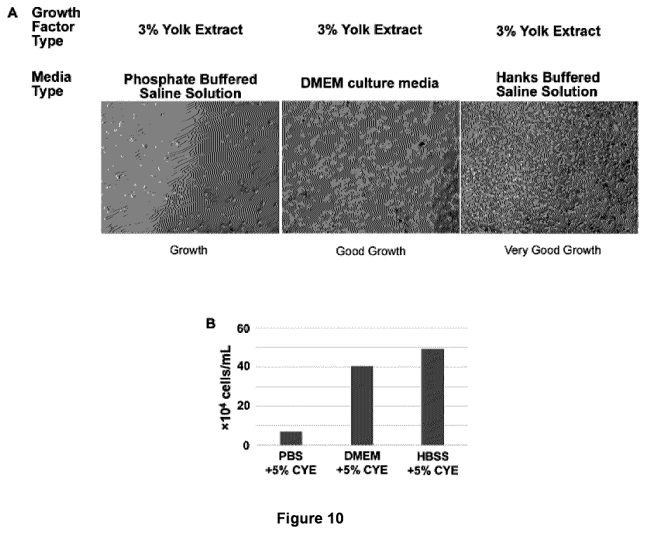

Due to the presence of glucose and amino acids within CYE, we performed experiments in which DMEM was replaced with alternative media, such as PBS (saline) and Hank's Buffered Saline Solution. These media alternatives lack amino acids and other key metabolites but could be a more economical solution if CYE is effective. In FIG. 10, we demonstrate that CYE can provide excellent growth conditions for MDA-MD-231 cells after 3 days in culture with HBSS compared to DMEM and PBS (FIG. 10A). When graphically presented, HBSS media was actually more effective than DMEM in terms of promoting cell proliferation over the 3 day timeframe.

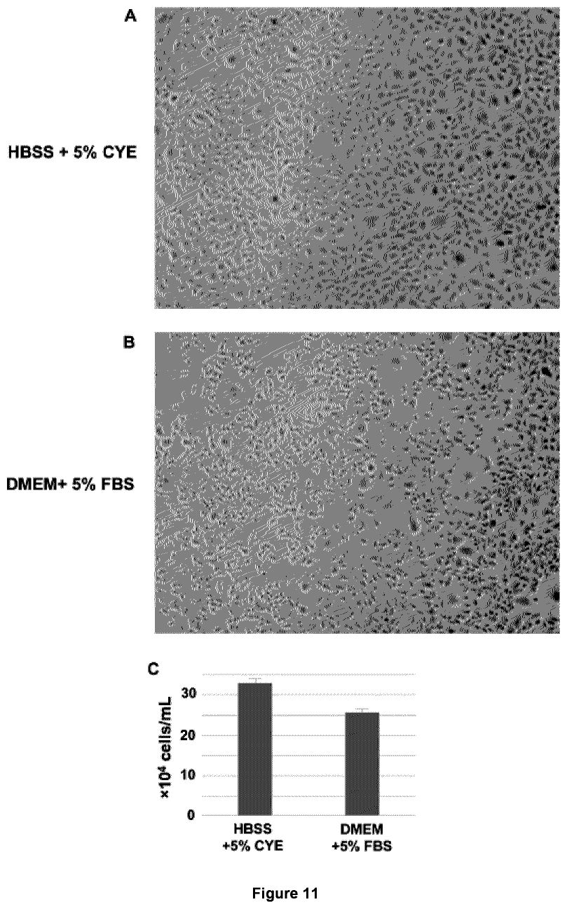

A head to head comparison was performed with conventional media that is used to normally culture MDA-MB-231 cells versus a superior formulation that used HBSS and CYE. The conventional media was DMEM+5% FBS versus our formulation which was HBSS+5% CYE. In FIG. 11A, the cells cultured with HBSS+5% CYE exhibited a near confluent monolayer of cells. In FIG. 11B, DMEM+5% FBS also lead to near confluent monolayer of cells. When cells were counted after 3 days of growth, HBSS+5% CYE culture media produced higher growth rates than HBSS+5% FBS (FIG. 11C). This suggests that CYE is able to produce similar or higher growth effects than conventional media supplemented by FBS.

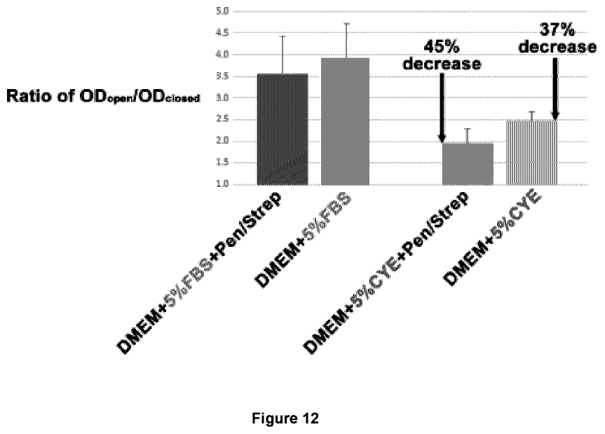

Egg white is known to contain various proteins that have anti-bactericidal properties, which is why eggs seldom are contaminated. The inclusion of egg white and its anti-bactericidal properties in the CYE was tested by exposing plates filled with culture media to ambient air overnight (FIG. 12). DMEM+5% FBS culture media was equally contaminated regardless of the presence of Pen/Strep (far left two bars). However, the use of DMEM+5% CYE revealed a significant decrease in the amount of bacterial load (decreased OD relative to their respective controls; far right two bars). This suggests that CYE at this concentration (5%) can result in decreased bacterial contamination in culture media.

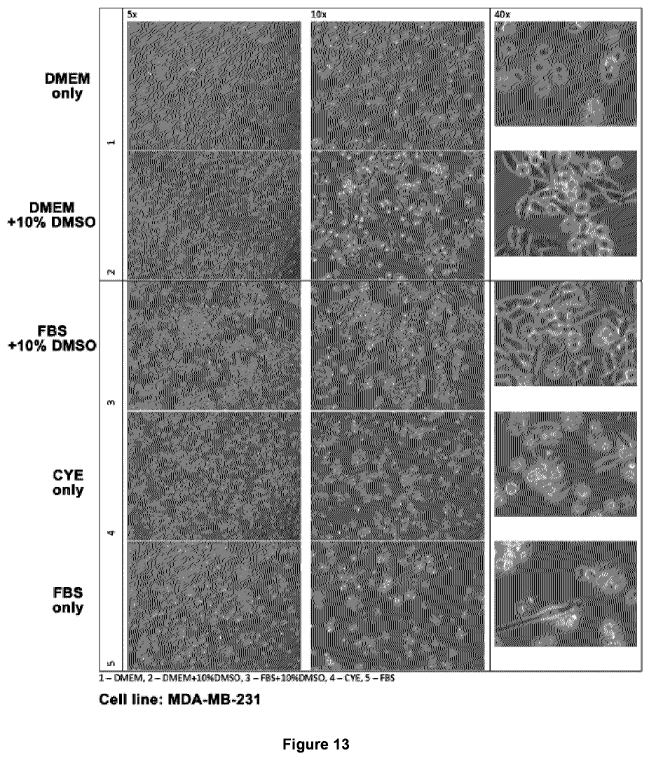

Due to the high amount of protein and lipid present within CYE, it was tested to see if it could act as a freezing media solution such as DMSO. DMSO is an effective freezing medium but upon thawing, cells will eventually die due to the presence of DMSO. In FIG. 13, CYE was compared to DMSO and FBS freezing medium. As predicted, the use of DMEM alone resulted in poor cell viability upon thawing (top row of images). Also, the use of DMEM+DMSO (10%) resulted in very high cell viability upon thawing, as expected (second top row of images). The use of FBS+DMSO (10%) was also effective and lead to adequate levels of cell viability post-thawing (middle row of images). However, CYE only (no DMSO, fourth row of images) led to high cell viability post-thawing and the cells appeared to be adherent. In contrast, FBS only did not result in high cell viability post-thaw and was as poor as DMEM alone (bottom row of images). These results show that CYE alone can be used as a freezing media and precludes the need for DMSO (10%) which can be toxic to cells under long-term culture conditions.

### CONCLUSIONS

The Complete Yolk Extract (CYE) described herein is a liquid product that is a suitable replacement for FBS in the culture of mammalian cells. It is composed of egg white (albumin) and egg yolk homogenates and made using method steps such as sonication, high pressure homogenization, and the addition of minute amounts of strong base to the egg mix. There is no dilution step involved in this production process. By using this process, the CYE is able to be filtered properly without clogging, which is important for sterility of the product and clarity of the supplemented cell culture media. In terms of performance, it is superior in terms of cell proliferation rates when compared to the same volume of FBS used. Moreover, it can be used to replace DMEM as well since it contains an abundance of amino acids, glucose, vitamins and other proteins that are normally part of a DMEM media recipe. In this manner, all that is needed is a buffered saline solution such as HBSS (Hanks Buffered Saline Solution) that is supplemented with 1-5% CYE to culture any cell of interest. This would also preclude the need to use pre-made specialized media such as DMEM or RPMI.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

What is claimed is:

1. An egg yolk extract comprising free yolk granules.

2. An egg yolk extract comprising the liquid contents of an egg yolk and one or more optionally lysed yolk granules.

3. An egg yolk extract comprising the liquid contents of yolk spheres.

4. An egg yolk extract comprising the liquid contents of yolk granules.

5. An egg yolk extract comprising the liquid contents of an egg yolk and one or more egg white proteins.

6. The egg yolk extract of claim 5, wherein the one or more egg white proteins comprise ovalbumin, ovotransferrin, lysozyme, ovalbumin-related protein X (OVAX), defensins, ovoinhibitor, AvBD11, or a combination thereof.

7. An egg yolk extract that supports cell proliferation and/or survival at least equivalent to FBS when used at the same concentration in a cell culture medium.

8. The egg yolk extract of any one of claims 2 to 7, comprising free yolk granules

9. The egg yolk extract of any one of claims 1 to 8, comprising lysed yolk spheres.

10. The egg yolk extract of any one of claims 7 to 9, comprising one or more egg white proteins such as ovalbumin, ovotransferrin, lysozyme, ovalbumin-related protein X (OVAX), defensins, ovoinhibitor, AvBD11, or a combination thereof.

11. The egg yolk extract of any one of claims 1 to 10, comprising the homogenized yolk and white of an egg.

12. The egg yolk extract of any one of claims 1 to 11, wherein the egg yolk extract consists essentially of egg yolk and egg white, wherein the egg yolk comprises lysed yolk spheres.

13. The egg yolk extract of any one of claims 1 to 12, wherein the egg yolk extract is liquid.

14. The egg yolk extract of any one of claims 1 to 13, wherein the egg yolk extract is extracted from an egg of any oviparous species, for example, birds, reptiles, amphibians, fish, insects, molluscs, arachnids, or combinations thereof.

15. The egg yolk extract of any one of claims 1 to 13, wherein the egg yolk extract is avian.

16. The egg yolk extract of claim 15, wherein the egg yolk extract is extracted from an egg of a chicken, turkey, duck, goose, quail, pheasant, ostrich, emu, or combinations thereof.

17. The egg yolk extract of claim 16, wherein the egg yolk extract is chicken, duck, or quail.

18. A supplement for cell culture medium, the supplement comprising the egg yolk extract of any one of claims 1 to 17.

19. The supplement of claim 18, consisting essentially of the egg yolk extract of any one of claims 1 to 17.

20. The supplement of claim 19, consisting of the egg yolk extract of any one of claims 1 to 16.

21. The supplement of any one of claims 1 to 20, wherein the yolk extract is not diluted.

22. The supplement of claim 21, wherein the yolk extract is not diluted with saline, such as PBS.

23. A cell culture medium comprising the egg yolk extract of any one of claims 1 to 16 or the supplement of any one of claims 18 to 22.

24. The cell culture medium of claim 23, wherein the medium is Dulbecco's Modified Eagle Medium (DMEM), RPMI media, CMRL1066, Hanks' Balanced Salt Solution (HBSS) phosphate buffered saline (PBS), L-15 medium, DMEM-F12, EpiLife® medium, and Medium 171, or a combination thereof.

25. The cell culture medium of claim 24, wherein the medium is a buffered saline solution, such as HBSS.

26. The cell culture medium of any one of claims 23 to 25, comprising from about 1% to about 10% v/v of the extract or supplement, such as about 1%, about 2%, about 3%, about 4%, about 5%, about 6%, about 7%, about 8%, about 9%, or about 10%.

27. The cell culture medium of any one of claims 23 to 26, wherein the cell culture medium does not contain FBS and/or NCS.

28. The cell culture medium of any one of claims 23 to 27, wherein the cell culture medium is a growth medium or a freezing medium, for example a freezing medium without DMSO.

29. The cell culture medium of any one of claims 23 to 28, wherein the cell culture medium is for culturing primary cell lines, for example, primary cell lines for food consumption, for example, primary bovine skeletal muscle cells.

30. The cell culture medium of any one of claims 23 to 29, wherein the cell culture medium is for use in culturing cells for antibody production.

31. The cell culture medium of any one of claims 23 to 29, wherein the cell culture medium is for use in biologics production, for example, biologics such as insulin, erythropoietin (EPO), or Granulocyte Colony Stimulating Factor (C-GSF).

32. A method for making an egg yolk extract, the method comprising lysing yolk spheres.

33. The method of claim 32, wherein lysing yolk spheres comprises sonicating or homogenizing the yolk spheres, for example by high pressure homogenization.

34. The method of claim 33, wherein the method is sufficient to lyse at least about 50%, about 60%, about 70%, about 80%, about 90%, about 95%, or about 99% of the yolk spheres.

35. The method of claim 33 or 34, wherein the sonicating is at about 75 W or more for from about 25 to about 30 cycles at about 15 to about 20 seconds per cycle.

36. The method of any one of claims 32 to 35, wherein the yolk spheres are in a composition comprising egg yolk and egg white.

37. The method of claim 36, further comprising mixing the egg yolk and egg white before lysing the yolk spheres, optionally wherein the mixing is for from about 30 minutes to about 24 hours, at from about 4° C. to about 25° C., such as for about 30 minutes at room temperature or for about 12 to 18 hours at 4° C., and optionally wherein the mixing is by inversion.

38. The method of claim 36 or 37, further comprising removing the vitelline membrane from the egg yolk before or after mixing the egg yolk and egg white.

39. The method of any one of claims 32 to 38, further comprising dissolving unlysed yolk spheres and/or yolk granules for example by alkalinizing and/or salinating the lysed yolk spheres.

40. The method of claim 39, wherein alkalinizing and/or salinating the lysed yolk spheres comprises adding a base and/or salt to the lysed yolk spheres, such as a strong base, such as NaOH (optionally at from about 100 mM to about 1 M), KOH (optionally at from about 100 mM to about 1 M), Na₂SO₄ (optionally at from about 0.05 mM to about 0.1 M), (NH₄)₂SO₄ (optionally at from about 0.05 mM to about 0.5 M), or a combination thereof or such as NaCl, optionally at from about 0.1 M to about 0.5M.

41. The method of any one of claims 32 to 40, further comprising removing solids.

42. The method of claim 41, wherein removing solids comprises centrifuging the lysed yolk spheres, for example at from about 15,000 g to about 30,000 g, and retaining the supernatant.

43. The method of any one of claims 32 to 42, further comprising sterilizing the egg yolk extract, for example, using a filter with a pore size of 0.4 μm or smaller, such as about a 0.22 μm or 0.11 μm pore size.

44. The method of any one of claims 32 to 43, further comprising clarifying the egg yolk extract, for example by adding glycerol at from about 50 to about 200 μL per 1 mL of egg yolk extract.

45. An egg yolk extract made by the method of any one of claims 32 to 44.
